(19) United States
(12) Patent Application Publication
(10) Pub. No.: US 2007/0179388 A1
(43) Pub. Date: Aug. 2, 2007

(54) Methods and systems of implanting a medical implant for improved signal detection

(76) Inventors: Vincent Larik, Kerkrade (NL); Fredric W. Lindemans, Sittard (NL)

Correspondence Address: MEDTRONIC, INC., 710 MEDTRONIC PARKWAY NE, MINNEAPOLIS, MN, 55432-9924, US

(21) Appl. No.: 11/343,073

(22) Filed: Jan. 30, 2006

(51) Int. Cl. A61N 1/00

(52) U.S. Cl. 600/508; 607/119

(57) ABSTRACT

A device and method of implanting a device for cardiac monitoring including a device can with one or more electrodes and one or more conductive bodies with one or more electrodes. The device may be implanted by measuring sequential surface ECG signals and identifying locations which produce signals having large magnitudes. The preferred location for implantation may be selected from these locations and the device may be implanted subdermally or intramuscullaryly in a position such that the device electrodes approximately correlate with the preferred electrode locations.

### BACKGROUND

Many embodiments in the present disclosure relate to methods of implanting a medical device in a preferred location for monitoring cardiac signals.

In many patients with cardiac arrhythmias, the arrhythmic events only occur intermittently, making clinical detection difficult. In addition, they may be of short duration and sudden onset with little or no warning, and may occur only infrequently. Because of this, the heart rhythms of such patients must be monitored continuously, over extended periods of time, in order to record an arrhythmic event.

Patients may be monitored though external devices such as Holter monitors which record electrocardiograms (ECGs) though electrodes attached to the skin. Such devices can make recordings over periods of time from days to a week or more. However, they are bulky and must be toted around by the patient, thus interfering with the patient's normal life and making them impractical for long term use. In addition, they may limit physical activities and must be removed during activities such as showering. Patients may also complain of skin irritation. Because the monitors must be worn for extended periods of time, these patient annoyances may result in poor patient compliance, decreasing their usefulness.

A variety of internally implanted medical devices monitor heart rhythms, and the data they collect can be sent to external systems through telemetry. For example, pacemakers, ICDs and other heart stimulating devices have leads in the heart for capturing physiologic parameters, including the cardiac electrograms and electrocardiograms (ECGs). Such devices, in addition to performing therapeutic operations, may monitor and transmit cardiac electrical signals (e.g., intracardiac electrocardiograms) to an external monitoring device, such as an external programmer, to allow the user to analyze the interaction between the heart and the implanted device. However, in patients who only need monitoring and do not require heart stimulation, the expense and risk from implanting an intracardiac lead is something both patients and physicians would prefer to avoid.

Other implanted medical devices are specifically for monitoring ECGs. The electrodes of these devices may be located on the device can or may be located outside the can on one or more conductive bodies, such as leads, coupled to the can. These devices are implanted subdermally such that the electrodes are in non-touching proximity to the heart. For devices which have the electrodes on the device can, the relative positions and spacing between the electrodes is fixed and the maximum length of the vector is approximately that of the device can. The choice of possible vectors and the length of the vectors are critical. However, detecting signal components with low amplitudes, such as P waves, which represent atrial activation and which are therefore important in arrhythmia classification, may be very difficult or impossible.

### BRIEF DESCRIPTION OF THE DRAWINGS

The following drawings are illustrative of particular embodiments of the present invention and therefore do not limit the scope of the invention. The drawings are intended for use in conjunction with the explanations in the following detailed description. Embodiments of the present invention will hereinafter be described in conjunction with the appended drawings, wherein like numerals denote like elements.

FIG. 1 is an illustration of an embodiment of the invention.

FIG. 2 is a block diagram of a main circuit and assembly of a device according to one embodiment.

FIG. 3 is an illustration of a tool for insertion of a device according to embodiments of the invention.

FIG. 4 is a block diagram of a method of implanting a device according to embodiments of the invention.

FIG. 5 is a device according to an embodiment located within a patient body segment.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

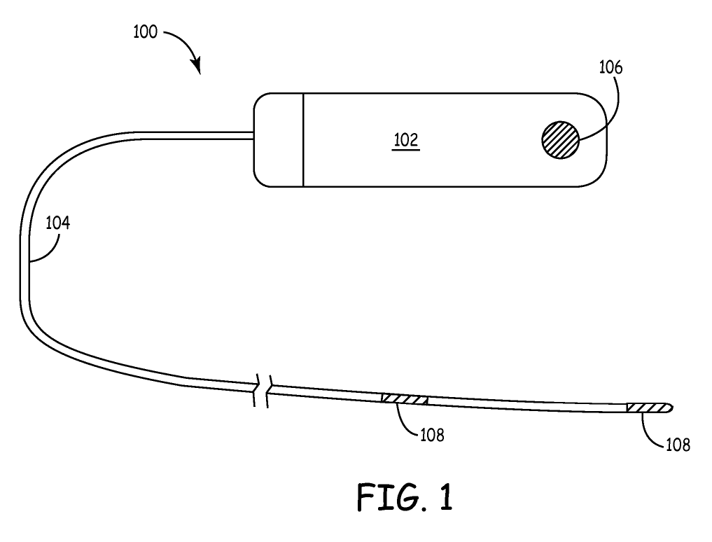

Referring to FIG. 1, the device 100 is an implantable electrode sensing system employing subcutaneous or intramuscular electrodes. Electrodes on the device sense myocardial potential differences generated by propagation of electrical activation of the heart cells in order to monitor cardiac activity. The device 100 includes a device can 102 that contains circuitry and includes at least one conductive body 104 extending from the can and electrically coupled to the components within the can. One or more electrodes 106 is located on the device can and one or more electrodes 108 is on the conductive body 104. The distance separating the electrodes on the conductive body from the device can allows for the sensing over enlarged vectors.

The device can 102 may have a variety of shapes. Preferably it is small to allow for easy implantation and minimal discomfort, once the preferred location is identified. For example, the device may be flat and thin with sides that are either straight, curved or both. For example, the device can 102 may be flat with a shape that is generally oval or generally rectangular. A rectangular device can could have sides which are straight, convex (wider on the ends than in the middle), or concave (wider in the middle than on the ends). Alternatively, one side of the device can could be concave and the opposite side could be convex such that the device can has a curved shape. In some embodiments the can may be tapered at one end. As described further below, the can includes one or more electrodes that may be portions of or all of the can itself or appendages extending locally from the can.

The conductive body 104 is flexible and extends from the device can to the desired location. It may contain one electrode 108 or may contain more than one electrode. For example, the conductive body may have a single tip electrode 108. Alternatively, it may have a tip electrode and one or more ring electrodes spaced along the length of the conductive body. An example of a suitable conductive body is the Medtronic heartwire model numbers 6491, 6494, 6495 and 6500. Any thin, flexible, unipolar, bipolar or multipolar conductive body, such as a pacing lead, might also be appropriate.

In one embodiment, the exterior of the device can is composed of a biocompatible material such as titanium or other metal or alloy. The device can may be coated with a compatible insulator but with exposed areas at the can electrodes. One or more electrodes on the device can provide electrical and physical surface contacts to the subdermal or intramuscular area of the body where the electrodes were placed. In certain embodiments, a flexible conductive body containing one or more electrodes which also provide surface contacts may also be attached to the device, such as by a sealed connector block.

The electrodes may be electrically connected via a feedthrough to a circuit board. The circuit board may contain all the electronics required for the device to function and is connected to a battery for power. An integrated circuit houses circuitry and intelligence required for the function and the memory is packaged on the other side of the circuit board. The device may include communications circuitry having a telemetry antenna both to indicate from outside the body that a read out is requested of the device, and for communicating data out from the device. Programming of the device or mode setting may also use the communications circuit. The components and functions of an implantable medical device for monitoring cardiac signals are disclosed in further detail in U.S. Pat. No. 5,987,352, the relevant portions of which are incorporated by reference.

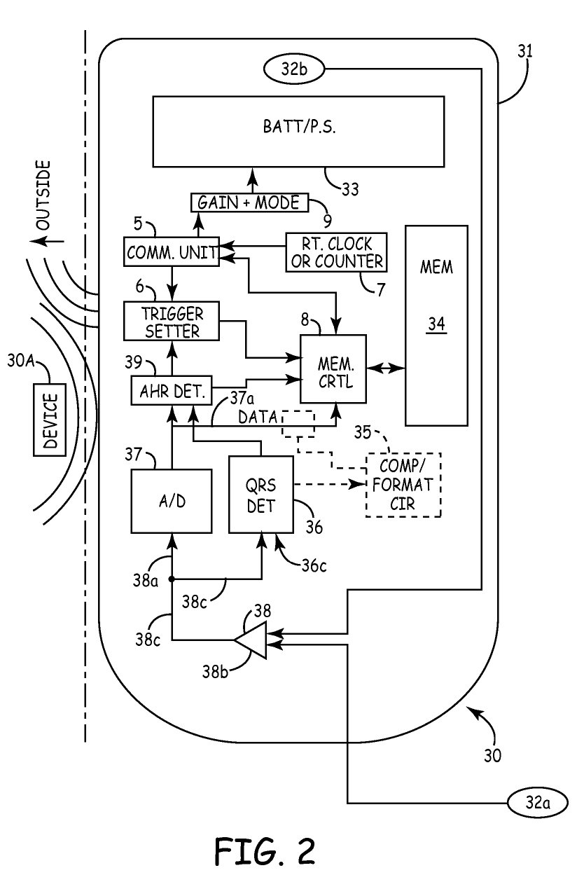

FIG. 2 is a circuit model according to an embodiment of the invention. Electrode 32a is located on a conductive body outside of the device can. The remaining components, including electrode 32b, are located on or within the device can.

In FIG. 2, a circuit model 30 is illustrated in an outline of an implantable device shell 31, electrodes 32a and 32b bring signal from the body to an input mechanism 38, here drawn as a differential amplifier for simplicity only, the output of which is fed to a QRS detector 36 and an A/D converter 37. Both these circuits 36 and 37 supply output to an arrhythmia detector 39, which in this preferred embodiment supplies the autotrigger signal to the trigger setting circuit 6. The data output from the analog to Digital converter may be converted, compressed, formatted and marked or reformulated if desired in a circuit 35 before the data is ready for input into the memory 34. The Memory control circuits 8 receives input from the A/D converter, with or without conversion and so forth from circuit 35, from the auto triggering determination circuit (here seen as the arrhythmia detection circuit) 39 (which may include input directly from the QRS detector if desired) as well as signals from the trigger setter circuit 6. The trigger setter circuit may also be controlled by a communications unit 5 which operates to receive and decode signals from the outside of the implant 30 that are telemetered or otherwise communicated in by a user. This communications unit 5 will also be able to communicate with the memory controller to request the offloading of memory data for analysis by an outside device. It should contain an antenna a or other transceiver device or circuitry to communicate with an outside device such as device 30A. A clock or counter circuit 7 reports the time since start or real time to the outside interrogator device 30A contemporaneously with a data offloading session so that the events recorded in memory 34 may be temporally pinpointed. Alternatives to this overall design may be considered, for example by using a microprocessor to accomplish some or all of the functions of circuits 6,8, 39, and 35. For a more detailed description of the components shown in FIG. 1, refer to U.S. Pat. No. 5,987,352, the relevant parts of which are hereby incorporated by reference.

In one embodiment, the device provides long term ECG monitoring of the subcutaneous (or intramuscular or submuscular) ECG. The device may continuously record and monitor the subcutaneous ECG in an endless loop of memory. The device may be triggered to save/retain a certain number of minutes of ECG recording. The device may itself trigger this recording after interpreting the signal it is receiving. This is referred to as autotriggering. Alternatively or additionally, the patient may signal the device to save an ECG recording, such as in response to certain physical symptoms. This is referred to as patient triggering and may be carried out using a variety of methods, such as small handheld external device featuring RF communication, tapping the device a certain number of times or with a certain cadence, light or sonic activation through the skin, or other types of signals. The device may be set to record a certain number of autotriggered events and/or a certain number of patient triggered events. Additionally, the amount of pre-trigger and post-trigger recording time could be set. Other options may include a compression algorithm. The choice of which types of triggers to record, how many of them to record, the length of the recordings, and whether or not the data should be compressed, will depend upon the clinical situation. In some embodiments, the physician may have the ability set the device to function in different modes including these or other variables.

By improving signal quality and amplitude, the problem of inappropriate autotriggering can be reduced. For example, a larger amplitude signal is less likely to fall below a threshold which would be interpreted as asystole and would also be more easily distinguished from noise artifact. Similarly, the P wave, R wave and T wave will be easier to identify given a higher quality signal. In addition, by selecting identical materials and sizes of the recording electrodes, the risk of signal loss artifact may be reduced. The ability of the physician to select locations for the electrodes to detect enlarged vectors according to various embodiments thus allows the unit to more correctly record arrhythmias by receiving high quality and large amplitude signals.

By placing one or more electrodes on a conductive body that may be implanted at a distance from the device can, the length of the vector or vectors over which the ECG or other cardiac signal may be detected may be increased. Because the vector or vectors may be extended, the morphological signal processing is improved. The improved signal processing allows for semi-permanent telemetry to external devices as compared to devices which are limited to detection over shorter vectors. By providing an electrogram signal having larger fidelity, P-wave recordings can be made with increased quality and improved reliability. The enlarged vector might also result in less critical placement of the device.

Because the conductive body is long and flexible, the physician is free to choose a location of the electrodes that provides the most desirable vector angle and length. The placement of the can and the length and flexibility of the conductive body allow for detection over vectors that are enlarged and are aligned for the desired magnitude and angle. By having a greater number of options for the subdermal or intramuscular locations of the electrodes, the likelihood of detecting a high quality signal is improved.

The preferred position of the device may be determined prior to implantation by using external ECG electrodes. By observing the ECG at various electrode orientations and positions in roughly the locations preferred by the physician and patient, the signal amplitudes of P wave, R wave and T wave can be monitored until good positioning is found and the signals are of an optimal amplitude. These measurements may also be made while the patient assumes different postures to account for posture variability. Because the distance between the electrode or electrodes of the can and those of the conductive body is not fixed, the testing electrodes which simulate those locations may be positioned with a range of distances between them, from closely adjacent to a distance greater than the length of the can, to as far apart as allowed when the conductive body is extended to its fullest length. In some embodiments, the device will include more than two electrodes, for example when either the device can, the conductive body, or both, contain more than one electrode. In such cases, if the distance between two or more electrodes is fixed, testing may be performed using spacing that approximates the distance between these electrode as well as the variable position electrode or electrodes. In addition, testing electrodes with approximately the same diameter as the device are appropriate.

Surface ECG measurements may be performed in a variety of ways. For example, a standard ECG monitoring system may be used with standard electrodes. Alternatively, a hand held device including electrodes, such as raised electrodes, may be used to probe the surface of the patient's body at various locations. To allow for variability of the separation of the electrodes, the distance between the testing electrodes should not be fixed but should be able to extend to the greatest possible separation of the electrodes allowed for the device. In another alternative, an electrode patch including multiple electrodes may be applied to the patients skin. Vector arithmetic may be used to determine the best and largest signal. For example, the locations producing the largest P wave, R wave or T wave could be identified arithmetically. In addition, whichever system of measuring is used, the data may be stored in a memory, may be processed by a processing circuit to locate the optimal angle of positioning of the electrodes, and information may be supplied to the physician using a user interface. Examples of suitable devices and methods for measuring surface ECGs for locating the optimum electrode locations are disclosed in U.S. Pat. No. 6,496,715, the relevant parts of which are incorporated by reference.

Because of the flexibility of device positioning afforded by locating one or more electrodes on a conductive body, a wide variety of device locations may be selected. This not only allows for more options for obtaining an ideal ECG measurement, but also may provide more flexibility in choosing more comfortable or aesthetically preferred locations. For example, the device can may be implanted in a location that is less visible after implantation, such as in the abdominal or axillary regions. Other possible locations for the device can include in the abdominal area and over the sternum.

The necessary distance between the electrode or electrodes of the conductive body and the electrode or electrodes of the device can after implantation is not fixed but rather the device is able to accommodate a close or a distant separation between electrodes. If a shorter separation is desired than the maximum distance between the electrodes, the conductive body may be gathered or looped around the device can to reduce its length. Furthermore, the conductive body can follow a straight or a curved path to place the one or more electrodes of the conductive body in the desired location.

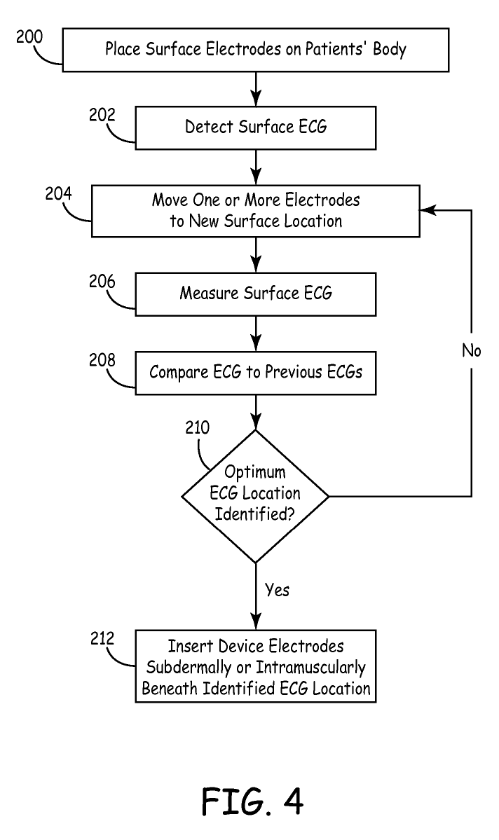

Once the preferred location of the electrodes is identified, the device is inserted subdermally. Alternatively it may be desirable to insert a portion or all of the device intramuscularly or submuscularly. The implantation procedure may be performed using a local anesthetic to numb the area of insertion of the device can as well as the pathway of the conductive body. A subdermal pocket is formed for placement of the device can, and a subdermal pathway is formed between the location of the device can and the desired location of the conductive body electrode or electrodes for insertion of the conductive body. The pocket and the pathway may be formed using an instrument such as a trocar. For example, the Medtronic Tunneling Tool Model 6996T is an appropriate tool for use in the implantation of the device. A method of implanting a device according to embodiments of the invention is illustrated in FIG. 4.

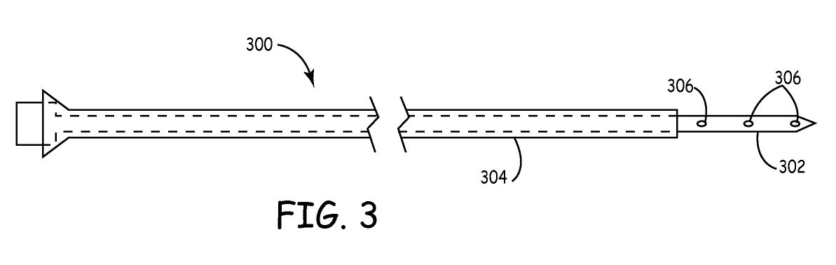

In one embodiment, the device 100 is inserted using a tool 300 including a long, thin needle 302 with a sheath 304 such as a plastic or metal sheath over its shaft. An example of such a needle 302 is shown in FIG. 3. The needle 302 may be inserted into the subcutaneous pathway, beginning in the pocket, and a local anesthetic may be infused through one or more holes 306 in the needle 302 while it is being advanced. The needle 302 may be pushed through the tissue slightly beyond the desired distal most position of the conductive body. As the needle 302 is subsequently withdrawn, the sheath 304 may remain in place, running from the pocket along the pathway of the conductive body. The sheath 304 may then serve as a tunnel through which the conductive body 104 may be advanced. After advancing the conductive body 104 through the sheath 304, the sheath 304 may then be removed. In some embodiments, the needle 302 may pierce outwardly through the skin surface at the distal end of the desired conductive body pathway to form a cutaneous exit site. In such embodiments, the sheath 304 may be withdrawn through the cutaneous exit site after the conductive body 104 is advanced into the sheath 304.

During the implantation procedure, the surgeon may wish to secure the device can and conductive body to the patient tissues such that the electrodes remain in the chosen locations. This may be done, for example, by using sutures, staples or a bioadhesive material. In one embodiment, suturing to hold the divide in place could be done automatically or with surgical staples by some means associated with the instrument. The device can and/or the conductive body could include holes or other features to aid in this attachment.

FIG. 4 illustrates an embodiment of the invention. In this embodiment, the can electrode is located on the device can and two conductive body electrodes, a ring electrode and a tip electrode, are located on the conductive body. In alternative embodiments, there could be a single electrode on the conductive body and one or more electrodes on the device can. In another embodiment, there could be two or more electrodes on the conductive body and no electrodes on the can. In other embodiments, there could be more than one conductive body, each of which could contain one or more electrodes. In embodiments including electrodes on more than one conductive body, there may or may not be an electrode located on the device can. A device containing two conductive bodies and no can electrodes may be preferred in certain situations for cosmetic reasons. For example, if the mapping of the desired location for the electrodes requires two electrodes to be placed relatively far apart but both on the chest wall, it may be preferable that both of these electrodes be provided by conductive bodies rather than a device can so that the device can may be placed in a less conspicuous location. In some embodiments, a device can electrode may be located on a short extensions from the device can, such as a stubby lead or fin as described in U.S. Pat. No. 5,987,352. This may be in addition to, or instead of, the device can electrode.

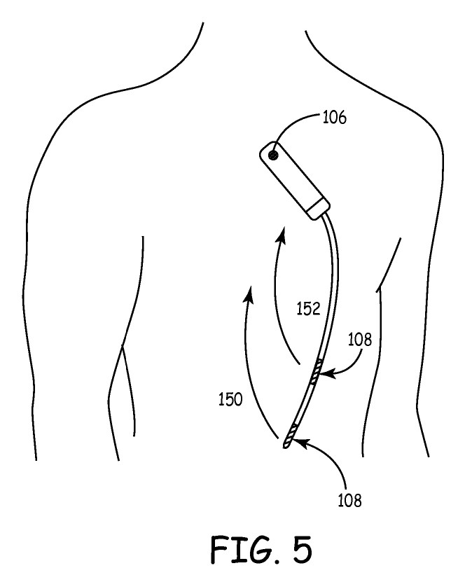

FIG. 5 illustrates a device implanted in a patient body according to an embodiment of the invention. The device includes a can electrode and two conductive body electrodes including a ring electrode and a tip electrode. As indicated by the arrows, the device may receive electrical signals from vectors 150, 152 between the tip electrode and the can electrode or between the ring electrode and the can electrode. By receiving data from two vectors, the device may be able to compare the data and provide more accurate interpretations of the signals. For example, the device might be better able to distinguish noise or signal loss from one electrode from a true event. Alternatively, the more than one electrode could each be located in positions which provide a preferred signal while the patient is in a different position, such as laying down and standing.

In the foregoing detailed description, the invention has been described with reference to specific embodiments. However, it may be appreciated that various modifications and changes can be made without departing from the scope of the invention as set forth in the appended claims.

1. A method of implanting an implantable device, wherein the device comprises a device can with one or more electrodes and a conductive body with one or more electrodes, the method comprising:
measuring sequential ECG signals using two or more testing electrodes on a surface of a patient's body,
identifying locations of the two or more testing electrodes which produce signals having large magnitudes,
selecting a preferred location for the device electrodes from the identified testing electrode locations,
subdermally or intramuscularly implanting the device such that the position of the device electrodes approximately correlates with the previously selected preferred electrode locations.

2. The method of claim 1, wherein the sequential ECG signals are measured using a standard ECG machine.

3. The method of claim 1, wherein the sequential ECG signals are measured using a hand held device having two or more testing electrodes, the distance between at least two of the testing electrodes being variable.

4. The method of claim 1, wherein the step of implanting the device is performed using a trocar.

5. The method of claim 1, wherein the signals having large magnitudes are R waves.

6. The method of claim 1, wherein the signals having large magnitude are P waves.

7. The method of claim 1, wherein the conductive body includes a tip electrode.

8. The method of claim 1, wherein the conductive body includes one or more ring electrodes.

9. The method of claim 1, wherein the preferred location is selected to minimize visibility of the device can after implantation within the patient's body.

10. The method of claim 9, wherein the preferred location of the device can is in the axillary region.

11. The method of claim 9, wherein the preferred location of the device can is in the abdominal region.

12. The method of claim 1, wherein the step of implanting the device comprises inserting an insertion tool comprising a needle and a sheath, subdermally or intramuscularly, and advancing the conductive body through the sheath.

13. An implantable medical device for detecting myocardial potential differences comprising:
a device can including one or more electrodes, and
a conductive body for subdermal or intramuscular implantation via an insertion tool, the conductive body extending from the device can and including one or more electrodes.

14. The implantable medical device of claim 13, wherein the conductive body is adapted for subdermal or intramuscular implantation via the insertion tool that includes a needle and a sheeth.

15. The implantable medical device of claim 13, wherein the conductive body electrode comprises one or more ring electrodes.

16. A system for detecting myocardial potential differences comprising:
an implantable medical device comprising a device can with one or more electrodes and a conductive body, extending from the device can, with one or more electrodes, and
an insertion tool for subdermal or intramuscular insertion of the conductive body.

17. The system of claim 16, further comprising two or more surface electrodes for detecting a surface ECG prior to insertion of the implantable medical device.

18. The system of claim 16, wherein the insertion tool comprises a needle for delivery of a local anesthetic.

19. The system of claim 16, wherein the insertion tool includes a sheath, the conductive body being advanceable within the sheath.

20. The system of claim 16, wherein the insertion tool includes a trocar.
